(19) United States
(12) Patent Application Publication
(10) Pub. No.: US 2026/0249041 A1
(43) Pub. Date: Aug. 27, 2026

(54) ELONGATED MEDICAL DEVICE AND METHOD FOR PRODUCING ELONGATED MEDICAL DEVICE

(71) Applicant: ASAHI INTECC CO., LTD., Seto-shi (JP)

(72) Inventors: Izumi KURIKI, Seto-shi (JP); Junji SATOZAKI, Seto-shi (JP); Taiji ITOU, Seto-shi (JP)

(73) Assignee: ASAHI INTECC CO., LTD., Seto-shi (JP)

(21) Appl. No.: 19/539,676

(22) Filed: Feb. 13, 2026

Related U.S. Application Data
(63) Continuation of application No. PCT/JP2024/027990, filed on Aug. 6, 2024.

(30) Foreign Application Priority Data
Aug. 18, 2023 (JP) 2023-133568

(51) Int. Cl. A61M 25/00

(52) U.S. Cl. CPC A61M 25/0009; A61M 2025/0046

(57) ABSTRACT

An elongated medical device including: an elongated body; and a swellable coating layer on at least a part of a surface of the elongated body. The swellable coating layer includes a first portion and a second portion that is disposed closer to a proximal end side of the elongated body than the first portion is to the proximal end side. The thickness of the first portion is greater than that of the second portion after the swellable coating layer is caused to swell by immersing the elongated medical device 1 in water at 37° C. for 120 minutes. The degree of swelling of the second portion is smaller than the degree of swelling of the first portion, where the degree of swelling is defined by equation (1):

Degree of swelling=(thickness after swelling)÷(thickness before swelling). (1)

### TECHNICAL FIELD

The disclosed embodiments relate to an elongated medical device and a method for producing the elongated medical device.

### BACKGROUND

Elongated medical devices such as a guide wire, a stent, and a catheter to be inserted into a living body are required to have lubricity on body tissues such as blood vessels in order to prevent the body tissues with which the elongated medical devices come into contact in the living body from being damaged, and to improve the operability of the elongated medical devices.

For this reason, it is a common practice to form a coating film made of a hydrophilic polymer and the like on a surface of the elongated medical device as described above. For example, Patent Literature 1 discloses an endoscope apparatus provided with a lubricating coating material formed such that the thickness on the side of a distal end portion is greater than the thickness on the side of a proximal end portion. Further, Patent Literature 2 discloses a guide wire provided with a lubricating resin coating having a density gradient of the lubricating resin such that frictional resistance decreases from a proximal end side to a distal end side.

### CITATION LIST

### Patent Literature

Patent Literature 1: Japanese Unexamined Patent Application Publication No. 2006-150040

Patent Literature 2: Japanese Unexamined Patent Application Publication No. 2006-192294

### SUMMARY

### Technical Problem

However, in the conventional elongated medical device provided with the above-described coating film, when the elongated medical device is inserted into a body cavity, friction is produced between the coating film and a body tissue, and a part of the coating film may be scraped off to generate fine particles.

An object of the disclosed embodiments is to provide an elongated medical device which exhibits excellent lubricity and is capable of suppressing generation of the fine particles.

### Solution to Problem

In order to achieve the above object, firstly, the disclosed embodiments provide an elongated medical device including: an elongated body which extends from a distal end side to a proximal end side; and a swellable coating layer which is provided on at least a part of a surface of the elongated body. The swellable coating layer includes a first portion and a second portion that is disposed closer to the proximal end side than the first portion is to the proximal end side. A thickness of the first portion is greater than a thickness of the second portion after the swellable coating layer is caused to swell by immersing the elongated medical device in water at 37° C. for 120 minutes; and a degree of swelling of the second portion is smaller than a degree of swelling of the first portion, where the degree of swelling is defined by equation (1):

Degree of swelling=(thickness after swelling)÷(thickness before swelling). (1)

### Effect

The elongated medical device according to the disclosed embodiments exhibits excellent lubricity and is capable of suppressing generation of fine particles.

### BRIEF DESCRIPTION OF DRAWINGS

FIG. 1 is a schematic cross-sectional view of an elongated medical device according to one embodiment of the disclosed embodiments.

FIG. 2 is a schematic cross-sectional view of an elongated medical device according to another embodiment of the disclosed embodiments.

FIG. 3 is a schematic cross-sectional view of an elongated medical device according to yet another embodiment of the disclosed embodiments.

### DETAILED DESCRIPTION

Embodiments of the disclosed embodiments will be described below.

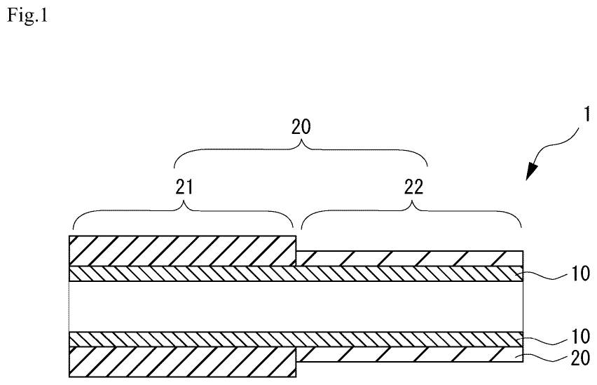

FIG. 1 shows a schematic cross-sectional view of an elongated medical device according to one embodiment of the disclosed embodiments. An elongated medical device 1 illustrated in FIG. 1 includes an elongated body 10, which extends from a distal end side (the left side in the drawing) to a proximal end side (the right side in the drawing), and a swellable coating layer 20 which is provided on at least a part of a surface of the elongated body 10. The swellable coating layer 20 includes a first portion 21 and a second portion 22 that is disposed closer to the proximal end side than the first portion 21. In the elongated medical device 1 shown in FIG. 1, the elongated body 10 is illustrated as a constituent element having a tubular form.

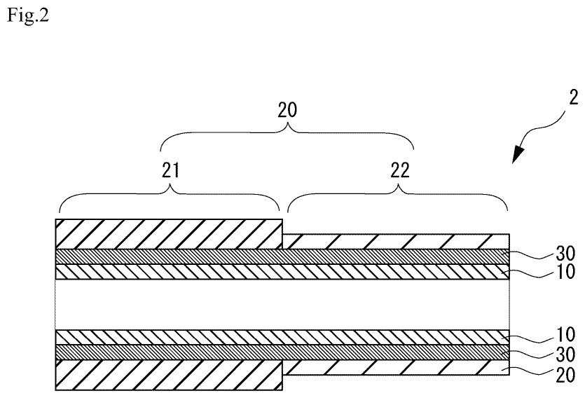

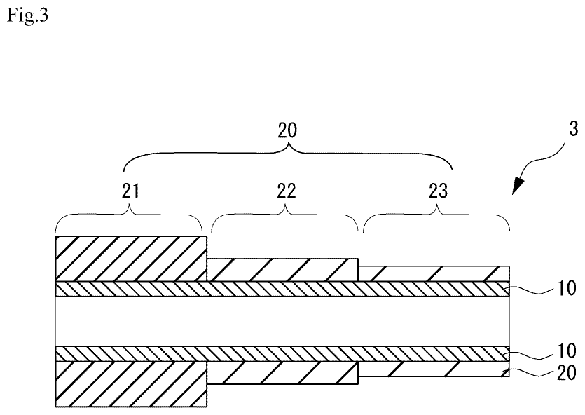

In the elongated medical device 1 according to the present embodiment, the thickness of the first portion 21 is greater than the thickness of the second portion 22 after the swellable coating layer 20 is caused to swell by immersing the elongated medical device 1 in water at 37° C. for 120 minutes. The elongated medical device 1 in FIG. 1 is depicted as being in a state in which the elongated medical device 1 has been immersed under the above-described conditions, and the swellable coating layer 20 is sufficiently swollen. The same applies to FIGS. 2 and 3, which will be described later.

Further, the degree of swelling of the swellable coating layer 20 defined by equation (1) below in the second portion 22 is smaller than the degree of swelling of the swellable coating layer 20 defined by equation (1) below in the first portion 21.

Degree of swelling=(thickness after swelling)÷(thickness before swelling). (1)

In the elongated medical device 1 according to the present embodiment, the thickness of the first portion 21 located on the distal end side is greater than the thickness of the second portion 22 located on the proximal end side. By this feature, the amount of water which contributes to lubrication is relatively increased at the distal end side, so that lubricity on the distal end side is improved and passability of the elongated medical device 1 through a body cavity is enhanced.

In addition, since the thickness of the second portion 22 is smaller than that of the first portion 21, contact between the swellable coating layer 20 and the body cavity is reduced at a position of the second portion 22 where lubricity is not so much required as compared with the distal end side. Moreover, since the degree of swelling of the second portion 22 is smaller than that of the first portion 21, in other words, the density of cross-linking of the second portion 22 is high, the strength of the second portion 22 is higher than that of the first portion 21. Thus, abrasion of the swellable coating layer 20 at the position of the second portion 22 is less likely to occur. Consequently, it becomes possible to suppress generation of fine particles from the swellable coating layer 20.

Furthermore, since the thickness of the second portion 22 is smaller than that of the first portion 21, lubricity of the swellable coating layer 20 is lowered on the proximal end side as compared with the distal end side. Thus, the elongated medical device 1 which is inserted into the body cavity can be easily supported (i.e., backup force is improved), and excellent operability can be obtained.

As described above, the elongated medical device 1 according to the present embodiment is excellent in lubricity on the distal end side, whereby the body cavity is less likely to be damaged, and fine particles are also less likely to be generated on the proximal end side.

The elongated medical device according to the disclosed embodiments is not limited to that illustrated in FIG. 1, and may have other forms and configurations. For example, FIG. 2 illustrates an elongated medical device 2 according to another embodiment. While the elongated medical device 2 illustrated in FIG. 2 includes an elongated body 10 and a swellable coating layer 20 likewise the elongated medical device 1, the elongated medical device 2 is further provided with a base layer 30 between the elongated body 10 and the swellable coating layer 20. As will be described later, the base layer 30 plays a role of improving adhesiveness of the swellable coating layer 20 to the elongated body 10. Moreover, the elongated medical device according to the disclosed embodiments may include the elongated body 10 and the swellable coating layer 20, and also include an adhesion layer provided therebetween. Likewise, the base layer 30, the adhesion layer also plays a role of improving the adhesiveness of the swellable coating layer 20 to the elongated body 10.

FIG. 3 illustrates an elongated medical device 3 according to yet another embodiment. While the elongated medical device 3 illustrated in FIG. 3 includes an elongated body 10 and a swellable coating layer 20 likewise the elongated medical device 1, the swellable coating layer 20 further includes, in addition to a first portion 21 and a second portion 22, a third portion 23 that is disposed closer to the proximal end side than the second portion 22. The thickness of the third portion 23 after swelling is smaller than the thickness of the second portion 22 after swelling. Further, the degree of swelling of the third portion 23 defined by the above equation (1) is smaller than that of the second portion.

As in the elongated medical device 1 illustrated in FIG. 1, the elongated medical devices 2 and 3 illustrated in FIGS. 2 and 3 are capable of suppressing the generation of fine particles while exhibiting excellent lubricity. Also, the third portion 23 may further be present in the elongated medical devices 1 and 2 illustrated in FIGS. 1 and 2. The elongated medical device 3 illustrated in FIG. 3 may also be provided with the base layer 30 or the adhesion layer. Further, in the elongated medical devices described above, there may exist a fourth portion (and fifth and subsequent portions) disposed closer to the proximal end side than the third portion.

### 1. Elongated Body

The elongated body 10 in the present embodiment is not particularly limited as long as it has a form extending from the distal end side to the proximal end side. The internal structure of the elongated body 10 may either be solid or hollow. Particularly, in the latter case, the elongated body 10 may be a tubular body whose internal structure includes a cavity extending in a longitudinal direction as illustrated in FIGS. 1 to 3.

The material of the elongated body 10 is not particularly limited, and may be selected as appropriate according to the elongated medical devices 1, 2, and 3 to be produced. Examples of the material include resin, metal, glass, and ceramic. Among these materials, resin is preferable.

Resin which constitutes the elongated body 10 is not particularly limited, and examples thereof include nylon resin such as polyamide, polyolefins such as polyethylene, polypropylene, and ethylene-propylene copolymer, polyester such as polyethylene terephthalate, thermoplastic resins such as polyvinyl chloride, ethylene-vinyl acetate copolymer, cross-linked ethylene-vinyl acetate copolymer, and polyurethane, polyamide elastomer, polyolefin elastomer, polyurethane elastomer, polystyrene elastomer, silicone rubber, latex rubber, and the like.

In the elongated medical devices 1, 2, and 3 according to the present embodiment, the first portion 21 and the second portion 22 may be differently formed by changing the resin which constitutes the elongated body 10 for a portion corresponding to the first portion 21, and a portion corresponding to the second portion 22 as will be described later. In this case, the elongated body 10 may include a first elongated body component disposed at a position where the first portion 21 is provided, and a second elongated body component disposed at a position where the second portion 22 is provided. When the swellable coating layer 20 includes the third portion 23 as in the case of the elongated medical device 3 illustrated in FIG. 3, the elongated body 10 may include a third elongated body component disposed at a position where the third portion 23 is provided. The first elongated body component, the second elongated body component, and the third elongated body component may be contiguous with each other directly or contiguous with each other indirectly.

As the material of the first to third elongated body components, the same kind of polymer may be used or different kinds of polymers may be used for each of these components. For example, even in a case where the same kind of polymer is used for each of the elongated body components, the degree of swelling of the elongated body components with respect to a solvent can be varied by adjusting the monomer component ratio or the like in the polymer. In this way, even in a case where the same kind of polymer is used, it is possible to form such an elongated body that the degree of swelling with respect to a specific solvent is different from the distal end side toward the proximal end side.

As described above, when the elongated body 10 is configured from the first elongated body component and the second elongated body component (and further the third elongated body component), the materials of the first to third elongated body components are not particularly limited. Polyurethane elastomer, polyolefin elastomer, polyester elastomer, polystyrene elastomer, polyamide elastomer or the like is preferable, and particularly, polyurethane elastomer or polyamide elastomer is preferable.

Even in the case of constituting the first elongated body component and the second elongated body component (and further the third elongated body component) by the same kind of resin, the first portion 21 and the second portion 22 (and further the third portion 23) can be differently formed by, for example, changing the composition ratio of the monomer constituting the resin.

### 2. Swellable Coating Layer

A material which constitutes the swellable coating layer 20 in the present embodiment is not particularly limited as long as the material can exhibit swellability and can constitute the first portion 21 and the second portion 22 described above.

In particular, the swellable coating layer 20 should preferably be constituted of a resin which exhibits swellability. As an example of the resin, a polymer having a reactive functional group (a hydroxyl group, a carboxyl group, an amino group, an epoxy group or the like) is preferable. Particularly, examples thereof include a polysaccharide-based polymer substance (a hyaluronic acid-based polymer substance, a cellulosic polymer substance, or the like), a polyethylene oxide-based polymer substance, a maleic acid-based polymer substance, an acrylamide-based copolymer, water-soluble nylon, polyvinyl alcohol, a polyvinylpyrrolidone-based copolymer, a hydroxyl group-containing acrylic copolymer, a known hydrophilic substance constituted of an amphoteric ion-based polymer substance having a hydrophilic group (e.g., a polymer having a betaine structure), and the like. Among the above examples, a hydroxyl group-containing acrylic copolymer should preferably be used. These polymers may be used alone, or two or more kinds of polymers may be combined and used.

Using a cross-linking agent together with the above-mentioned resin is also preferable. In particular, since the degree of swelling of the swellable coating layer 20 can be adjusted according to the degree of cross-linking of the above-mentioned polymers, the use of a cross-linking agent makes it possible to easily achieve a desired degree of swelling.

Examples of the cross-linking agent include an isocyanate-based cross-linking agent, an epoxy-based cross-linking agent, an amine-based cross-linking agent, a melamine-based cross-linking agent, an aziridine-based cross-linking agent, a hydrazine-based cross-linking agent, an aldehyde-based cross-linking agent, an oxazoline-based cross-linking agent, a metal alkoxide-based cross-linking agent, a metal chelate-based cross-linking agent, a metal salt-based cross-linking agent, and an ammonium salt-based cross-linking agent. The cross-linking agent may be used alone, or two or more kinds of cross-linking agents may be combined and used.

When the polymer described above has a hydroxyl group as a reactive group, an isocyanate-based cross-linking agent having excellent reactivity with the hydroxyl group should preferably be used as the cross-linking agent.

The isocyanate-based cross-linking agent contains at least a polyisocyanate compound. Examples of the polyisocyanate compound include aromatic polyisocyanates such as a tolylene diisocyanate, a diphenylmethane diisocyanate, and a xylylene diisocyanate; aliphatic polyisocyanates such as a pentamethylene diisocyanate and a hexamethylene diisocyanate; and alicyclic polyisocyanates such as an isophorone diisocyanate and a hydrogenated diphenylmethane diisocyanate, and their biurets, isocyanurates, and allophanates, and also, an adduct which is a product reacted with a low-molecular active hydrogen-containing compound such as ethylene glycol, propylene glycol, neopentyl glycol, trimethylolpropane, and castor oil. Among the above examples, hexamethylene diisocyanate should preferably be used.

When a cross-linking agent is used, the amount of the cross-linking agent used should preferably be 1 to 100 parts by mass with respect to 100 parts by mass of the resin described above. Particularly, the amount of the cross-linking agent should preferably be 5 to 80 parts by mass, and more preferably, 10 to 50 parts by mass with respect to 100 parts by mass of the resin described above.

A method for forming the swellable coating layer 20 in the present embodiment is not particularly limited. For example, the swellable coating layer 20 can be formed by mixing the above-described resin with a cross-linking agent, as desired, in a predetermined solvent to prepare a coating material, applying the coating material onto the elongated body 10 (the base layer 30 if the base layer 30 is present), and drying the coating material. Examples of a condition of the drying include performing the drying at a temperature in a range of room temperature to 150° C., or preferably, 40 to 100° C., for 1 to 1440 minutes, or preferably, 20 to 800 minutes.

Examples of the solvent include a mixed solvent of water and ethanol, water alone, ethanol alone, isopropanol, and the like. Among these examples, a mixed solvent of water and ethanol is preferable.

The thickness of the swellable coating layer 20 of the present embodiment before the swellable coating layer 20 is caused to swell should preferably be 0.1 to 100 μm. Particularly, the thickness should preferably be 0.5 to 20 μm, and more preferably, 1 to 10 μm. Before the swellable coating layer 20 is caused to swell, there may be no difference in the thickness between the first portion 21 and the second portion 22 (and the second portion 23, if present).

Meanwhile, after the swellable coating layer 20 of the present embodiment is caused to swell, the thickness of the first portion 21 should preferably be 1 to 200 μm. Particularly, the thickness of the first portion 21 should preferably be 5 to 100 μm, and more preferably, 10 to 50 μm. Further, the thickness of the second portion 22 should preferably be 0.5 to 100 μm. Particularly, the thickness of the second portion 22 should preferably be 2 to 80 μm, and more preferably, 3 to 30 μm. Furthermore, if the third portion 23 is present, the thickness thereof should preferably be 0.1 to 80 μm. Particularly, the thickness of the third portion 23 should preferably be 1 to 50 μm, and more preferably, 1 to 30 μm.

In the swellable coating layer 20 of the present embodiment, the degree of swelling of the swellable coating layer 20 defined by equation (1) below should preferably be 10 to 2000 for the first portion 21. Particularly, the degree of swelling for the first portion 21 should preferably be 13 to 200, and more preferably, 15 to 100. As for the second portion 22, the degree of swelling should preferably be 5 to 1000. Particularly, the degree of swelling for the second portion 22 should preferably be 7 to 800, and more preferably, 8 to 300. Further, if the third portion 23 is present, the degree of swelling should preferably be 1 to 800 for the third portion 23. Particularly, the degree of swelling for the third portion 23 should preferably 2 to 500, and more preferably, 3 to 300.

Degree of swelling=(thickness after swelling)÷(thickness before swelling). (1)

In the present specification, unless otherwise specified, the condition for causing the swellable coating layer 20 to swell refers to a condition for causing the swellable coating layer 20 to be sufficiently swollen by immersing the elongated medical devices 1, 2, and 3 in water at 37° C. for 2 hours.

### 3. Base Layer

A material which constitutes the base layer 30 in the present embodiment is not particularly limited as long as the base layer 30 can cause the swellable coating layer 20 and the elongated body 10 to adhere to each other. Preferred examples of the material include a (meth)acrylic resin, a polyvinyl alcohol resin, a polyhydroxyethyl methacrylate resin, a polyethylene glycol resin, an acrylic acid resin, and a maleic acid resin. It suffices that the (meth)acrylic resin is a resin having a polymerization unit based on a monomer having an acryloyl group (H₂C═CH—C(═O)—) or a methacryloyl group (H₂C═C(CH₃)—C(═O)—).

A cross-linking agent may be added to the above-described resins for constituting the base layer 30. The cross-linking agent may be an agent to cross-link the resin for constituting the base layer 30, or may be an agent to cross-link the resin for constituting the swellable coating layer 20. In the latter case, when a coating material for forming the swellable coating layer 20 is applied onto the base layer 30, the cross-linking agent transfers from the base layer 30 toward the coating material to cross-link the resin for constituting the swellable coating layer 20.

As an example of the cross-linking agent, the same cross-linking agent as that employed as the material of the swellable coating layer 20, for example, may be used. When a cross-linking agent is added to the resin for constituting the base layer 30, the amount of the cross-linking agent used should preferably be 1 to 100 parts by mass with respect to 100 parts by mass of the resin. Particularly, the amount of the cross-linking agent should preferably be 5 to 80 parts by mass, and more preferably, 10 to 50 parts by mass with respect to 100 parts by mass of the resin.

A method for forming the base layer 30 in the present embodiment is not particularly limited. For example, the base layer 30 can be formed by mixing the above-described resin with a cross-linking agent, as desired, in a predetermined solvent to prepare a coating material, applying the coating material onto the elongated body 10, and drying the coating material. Examples of a condition of the drying include performing the drying at a temperature in a range of room temperature to 150° C., or preferably, 40 to 100° C., for 1 to 1440 minutes, or preferably, 20 to 800 minutes.

Examples of the solvent include propylene glycol monomethyl ether acetate (PGMEA), ethyl acetate, butyl acetate, and toluene. Among these examples, PGMEA is preferable.

The thickness (before swelling) of the base layer 30 in the present embodiment should preferably be 0.1 to 100μm. Particularly, the thickness should preferably be 0.5 to 50 μm, and more preferably, 1 to 10 μm.

4. A material which constitutes the adhesion layer in the present embodiment is not particularly limited as long as the adhesion layer can cause the swellable coating layer 20 and the elongated body 10 to adhere to each other. However, the adhesion layer is different from the base layer 30 in that while the adhesion layer contains a cross-linking agent as a main material thereof, a polymer component is not contained as the main material.

As a preferred example of the cross-linking agent, the same cross-linking agent as that employed as the material of the swellable coating layer 20, for example, may be used.

A method for forming the adhesion layer in the present embodiment is not particularly limited. For example, the adhesion layer can be formed by mixing a cross-linking agent into a predetermined solvent to prepare a coating material, applying the coating material onto the elongated body 10, and drying the coating material. Examples of a condition of the drying include performing the drying at a temperature in a range of room temperature to 150° C., or preferably, 40 to 100° C., for 1 to 1440 minutes, or preferably, 20 to 800 minutes.

As an example of the solvent, the same solvent as that employed as the material of the base layer 30 may be used.

### 5. Elongated Medical Device

The type of the elongated medical devices 1, 2, and 3 according to the present embodiment is not particularly limited as long as the devices are required to have lubricity on body tissues. Generally, the elongated medical devices 1, 2, and 3 are suitably used as an elongated medical device which is inserted into or disposed within a living body, i.e., as a guide wire or a catheter, in particular.

Examples of the catheter are not particularly limited, and include a guiding catheter, a penetration catheter, a microcatheter, a balloon catheter, a foreign object removal catheter, a contrast imaging catheter, a biliary catheter, a urethral catheter, an endoscope, and a dilator. The catheter should preferably be provided with a tubular elongated body which is made of a resin and extends from a distal end side to a proximal end side, and a swellable coating layer which is provided on an outer periphery of the tubular elongated body, and the base layer described above may be provided between the tubular elongated body and the swellable coating layer. The resin forming the tubular elongated body where the first portion of the swellable coating layer is disposed may be different from the resin forming the tubular elongated body where the second portion is disposed.

Examples of the guide wire are also not particularly limited, and include a percutaneous coronary intervention (PCI) guide wire for treating coronary arteries, a percutaneous transluminal angioplasty (PTA) guide wire for treating lower limb blood vessels, an interventional radiology (IVR) guide wire for treating peripheral blood vessels, an INR guide wire for treating cerebral blood vessels, and a coronary angiography (CAG) guide wire for contrast imaging.

### 6. Method for Producing Elongated Medical Device

The elongated medical devices 1, 2, and 3 according to the present embodiment can be produced, for example, by preparing the elongated body 10, and sequentially layering the base layer 30 or the adhesion layer, if desired, and the swellable coating layer 20 on at least a part of the surface of the elongated body 10.

A method for differently forming the first portion 21 and the second portion 22 (and further the third portion 23) in the swellable coating layer 20 is not particularly limited. For example, the first portion 21 and the second portion 22 (and further the third portion 23) can be formed by preparing coating materials that contain the materials of these portions, respectively, and individually applying the coating materials onto the surface of the elongated body 10 (or the base layer 30).

While the first portion 21 and the second portion 22 (and further the third portion 23) are different from each other in the thickness and the degree of swelling when they are swollen as described above, these differences can be caused by changing the crosslinking density of the resin which constitutes the respective portions.

For example, when the swellable coating layer 20 is formed by using a coating material prepared by mixing a hydrophilic polymer and a cross-linking agent in a solvent, the number of cross-linking points can be increased by increasing the amount of the cross-linking agent with respect to the hydrophilic polymer. As a result, the degree of swelling can be reduced and the thickness of the swellable coating layer 20 when it is swollen can be reduced. In contrast, by reducing the amount of the cross-linking agent with respect to the hydrophilic polymer, the number of cross-linking points can be decreased. As a result, the degree of swelling can be increased and the thickness of the swellable coating layer 20 when it is swollen can be increased.

Therefore, while adding the same amount of hydrophilic polymer to the coating materials for forming the first portion 21 and the second portion 22, a smaller amount of cross-linking agent is added to the coating material for the first portion 21, and a larger amount of cross-linking agent is added to the coating material for the second portion 22. In this way, the crosslinking density of the first portion 21 can be reduced as compared with the second portion 22, and as a consequence, the first portion 21 can be formed such that the degree of swelling is more increased and the thickness of the first portion 21 when it is swollen is more increased.

Adjusting the amount of the cross-linking agent described above can also be realized by adjusting the swellability (absorbability) of the elongated body 10 (or the base layer 30) with respect to a solvent.

For example, by forming a portion (the first elongated body component) of the elongated body 10 where the first portion 21 is to be formed by using a resin that easily swells with respect to a solvent of the above-mentioned coating material, a cross-linking agent can be transferred into the resin together with the solvent from the coating material applied on that portion. As a result of this transfer, the amount of the cross-linking agent in the first portion 21 can be reduced. Meanwhile, by forming a portion (the second elongated body component) where the second portion 22 is to be formed by using a resin that is less likely to swell with respect to a solvent of the above-mentioned coating material, the above-described transfer of the cross-linking agent is suppressed. Thus, the amount of the cross-linking agent in the second portion 22 can be reduced.

From the standpoint that the above procedure enables the elongated medical devices 1, 2, and 3 according to the present embodiment to be more easily produced, the elongated medical devices 1, 2, and 3 should preferably be produced by a method which utilizes the adjustment of the amount of cross-linking agent by using the elongated body 10 as described above. A suitable example of such a production method is, for instance, a production method including:
a step of preparing an elongated body in which a first elongated body component (I) made of a first resin (I) and a second elongated body component (II), which is disposed on a proximal end side of the first elongated body component (I) and is made of a second resin (II) having a smaller degree of swelling with respect to a first solvent (A) than the first resin (I), are directly or indirectly contiguous with each other; and an application step of applying a coating material containing a cross-linking agent and the first solvent (A) onto at least a part of surfaces of the first elongated body component (I) and the second elongated body component (II).

In the above production method, one preferred aspect is that in the application step, a swellable coating layer should be formed by applying a coating material containing a hydrophilic polymer, a cross-linking agent, and the first solvent (A) onto at least a part of the surfaces of the first elongated body component (I) and the second elongated body component (II).

As for the elongated medical device 2 including the base layer 30, it is also possible to add a cross-linking agent to a coating material for forming the base layer 30 and cause the cross-linking agent to transfer into the coating material of the swellable coating layer 20, thereby adjusting the amount of the cross-linking agent in the swellable coating layer 20.

Also in this case, in the elongated body 10, a portion where the first portion 21 is to be formed is formed by a resin that easily swells with respect to a solvent, and a portion where the second portion 22 is to be formed is formed by a resin that is less likely to swell with respect to the solvent. In this way, it is possible to cause the cross-linking agent in the coating material for forming the base layer 30 to transfer into these resins, and produce, according to a difference in the amount of the transfer at that time, a difference in the amount of the cross-linking agents between the first portion 21 and the second portion 22 that are formed on the base layer 30, whereby a difference in the crosslinking density can be produced.

A suitable example of a method for producing the elongated medical device 2 including the base layer 30 is, for instance, a production method including:
a step of preparing an elongated body in which a first elongated body component (I) made of a first resin (I) and a second elongated body component (II), which is disposed on a proximal end side of the first elongated body component (I) and is made of a second resin (II) having a smaller degree of swelling with respect to a first solvent (A) than the first resin (I), are directly or indirectly contiguous with each other; and
a step of forming a base layer by applying a coating material containing a base polymer, a cross-linking agent, and the first solvent (A) onto at least a part of surfaces of the first elongated body component (I) and the second elongated body component (II); and
a step of forming a swellable coating layer by applying a coating material containing a hydrophilic polymer and a second solvent (B) onto at least a part of a surface of the base layer on a side opposite to the elongated body.

As for the elongated medical device including an adhesion layer, as in the elongated medical device 2 including the base layer 30, it is also possible to add a cross-linking agent to a coating material for forming the adhesion layer, and cause the cross-linking agent to transfer into the coating material of the swellable coating layer 20, thereby adjusting the amount of the cross-linking agent in the swellable coating layer 20.

A suitable example of a method for producing the elongated medical device including the adhesion layer is, for instance, a production method including:
a step of preparing an elongated body in which a first elongated body component (I) made of a first resin (I) and a second elongated body component (II), which is disposed on a proximal end side of the first elongated body component (I) and is made of a second resin (II) having a smaller degree of swelling with respect to a first solvent (A) than the first resin (I), are directly or indirectly contiguous with each other; and
a step of forming an adhesion layer by applying a coating material containing a cross-linking agent and the first solvent (A) onto at least a part of surfaces of the first elongated body component (I) and the second elongated body component (II); and
a step of forming a swellable coating layer by applying a coating material containing a hydrophilic polymer and a second solvent (B) onto at least a part of a surface of the adhesion layer on a side opposite to the elongated body.

The coating material for forming the swellable coating layer 20, the coating material for forming the base layer 30, and the coating material for forming the adhesion layer can be applied by a general method.

The embodiments explained above are described to facilitate understanding of the disclosed embodiments and not to limit the disclosed embodiments. Thus, the elements disclosed in the above-described embodiments are intended to include all design changes and equivalents belonging to the technical scope of the disclosed embodiments.

### Examples

The disclosed embodiments will be more specifically explained with reference to Examples and the like below, but the scope of the disclosed embodiments is not limited to the following Examples and the like.

### Experimental Example 1

### (1) Confirmation of Swellability

A resin plate made of ether-based polyurethane elastomer was cut into a size of 10 mm in length, 10 mm in width, and 0.1 mm in thickness, and used as a first resin plate. When the first resin plate was immersed in propylene glycol monomethyl ether acetate (PGMEA) overnight in an environment of 23° C., the length and the width increased by 1.4 times.

Further, a resin plate made of polyamide elastomer was cut into a size of 10 mm in length, 10 mm in width, and 0.1 mm in thickness, and used as a second resin plate. When the second resin plate was immersed in PGMEA overnight in an environment of 23° C., substantially no swelling occurred.

### (2) Sample Production

A coating material for a base layer was prepared by mixing 10.58% by mass of a hydrophobic acryl polyol resin and 3.63% by mass of hexamethylene diisocyanate, as a cross-linking agent, in 85.79% by mass of PGMEA as a solvent.

Further, a coating material for a swellable coating layer was prepared by dissolving 10% by mass of a water-soluble hydroxyl group-containing acrylic resin in 90% by mass of a mixed solvent of water and ethanol.

Subsequently, the coating material for a base layer, which has been prepared as described above, was applied onto a resin tube made of an ether-based polyurethane elastomer resin described above and dried by heating at 60° C. for 30 minutes, thereby forming a base layer having a thickness of 4 μm.

Further, the coating material for a swellable coating layer, which has been prepared as described above, was applied onto the formed base layer and dried by heating at 70° C. for 90 minutes, thereby forming a swellable coating layer having a thickness of 1 μm.

In this way, a first sample in which the base layer and the swellable coating layer are sequentially layered on the resin tube made of the ether-based polyurethane elastomer resin was obtained.

Further, a base layer and a swellable coating layer were formed in the same manner as those in the first sample except that the resin tube made of the ether-based polyurethane elastomer resin was changed to a resin tube made of a polyamide elastomer resin described above. In this way, a second sample in which the base layer and the swellable coating layer are sequentially layered on the resin tube made of the polyamide elastomer resin was obtained.

### Test Example 1

### Measurement of Amount of Cross-Linking Agent on Surface of Base Layer

During a process of producing the first sample and the second sample as described above, the amount of cross-linking agent on a surface of the base layer was measured by Fourier transform infrared spectrometry (FT-IR method) immediately after the base layer has been formed. As a result, it was found that the amount of the cross-linking agent in the second sample was greater than that of the first sample by 1.2 times.

### Test Example 2

### Measurement of Thickness of Swellable Coating Layer After Swelling

The thickness of the swellable coating layer after swelling was measured for the produced first sample and second sample. Specifically, the first sample and the second sample were immersed in water at a temperature of 27° C. for 120 minutes to cause their respective swellable coating layers to be sufficiently swollen. Then, the thicknesses of the swellable coating layers after swelling were measured.

As a result, in the first sample, the thickness of the swellable coating layer after swelling was 34 μm. On the other hand, in the second sample, the thickness of the swellable coating layer after swelling was 12 μm. That is, in the first sample, the thickness of the swellable coating layer after swelling was approximately three times that of the second sample.

Also, when the degree of swelling of the swellable coating layer was calculated for each of the first sample and the second sample on the basis of equation (1) below, the degree of swelling was 34 for the first sample and 12 for the second sample.

Degree of swelling=(thickness after swelling)÷(thickness before swelling). (1)

From the results of the Test Examples described above, it has been found that varying the materials of the resin tubes causes the amounts of the cross-linking agent in the base layers to be changed, and also makes it possible to change the thickness of the swellable coating layer after swelling and the degree of swelling.

Therefore, it is understood that the elongated medical device according to the present embodiment can be produced by forming the first portion in the same manner as that in the first sample, and forming the second portion in the same manner as that in the second sample.

In order to achieve the above objects, firstly, the disclosed embodiments provide an elongated medical device including: an elongated body which extends from a distal end side to a proximal end side; and a swellable coating layer which is provided on at least a part of a surface of the elongated body. The swellable coating layer includes a first portion and a second portion that is disposed closer to the proximal end side than the first portion is to the proximal end side. A thickness of the first portion is greater than a thickness of the second portion after the swellable coating layer is caused to swell by immersing the elongated medical device in water at 37° C. for 120 minutes; and a degree of swelling of the second portion is smaller than a degree of swelling of the first portion, where the degree of swelling is defined by equation (1):

Degree of swelling=(thickness after swelling)÷(thickness before swelling). (1)

### Embodiment 1

In the above embodiment (Embodiment 1), preferably, the swellable coating layer includes a third portion that is disposed closer to the proximal end side than the second portion is to the proximal end side. The thickness of the second portion after the swelling is greater than a thickness of the third portion after the swelling, and a degree of swelling, defined by the equation (1), of the third portion is smaller than the degree of swelling the second portion. (Embodiment 2)

Secondly, the disclosed embodiments provide a method for producing an elongated medical device including an elongated body which extends from a distal end side to a proximal end side. The method includes: a step of preparing the elongated body in which a first elongated body component (I) and a second elongated body component (II), which is disposed on a proximal end side of the first elongated body component (I), are directly or indirectly contiguous with each other. The first elongated body component (I) is made of a first resin (I), and the second elongated body component (II) is made of a second resin (II) having a smaller degree of swelling with respect to a first solvent (A) than the first resin (I). The method further includes an application step of applying a first coating material containing a cross-linking agent and the first solvent (A) onto at least a part of surfaces of the first elongated body component (I) and the second elongated body component (II).

### Embodiment 3

In the above embodiment (Embodiment 3), preferably, in the application step, a swellable coating layer is formed by applying the first coating material, and the first coating material further includes a hydrophilic polymer. (Embodiment 4)

In the above embodiment (Embodiment 3), it is also preferable that in the application step, a base layer is formed by applying the first coating material, which further includes a base polymer, and the method further includes a step of forming, after the application step, a swellable coating layer by applying a second coating material containing a hydrophilic polymer and a second solvent (B) onto at least a part of a surface of the base layer on a side opposite to the elongated body. (Embodiment 5)

In the above embodiment (Embodiment 3), it is also preferable that in the application step, an adhesion layer is formed by applying the first coating material, and the method further includes a step of forming, after the application step, a swellable coating layer by applying a second coating material containing a hydrophilic polymer and a second solvent (B) onto at least a part of a surface of the adhesion layer on a side opposite to the elongated body. (Embodiment 6)

### INDUSTRIAL APPLICABILITY

The elongated medical device according to the disclosed embodiments is suitable as a guide wire or a catheter, for example.

### DESCRIPTION OF REFERENCE NUMERALS

1, 2, 3 Elongated medical device
10 Elongated body
20 Swellable coating layer
21 First portion
22 Second portion
23 Third portion
30 Base layer

1. An elongated medical device comprising:
an elongated body which extends from a distal end side to a proximal end side; and
a swellable coating layer which is provided on at least a part of a surface of the elongated body, wherein:
the swellable coating layer includes a first portion and a second portion that is disposed closer to the proximal end side than the first portion is to the proximal end side;
a thickness of the first portion is greater than a thickness of the second portion after the swellable coating layer is caused to swell by immersing the elongated medical device in water at 37 °C for 120 minutes; and
a degree of swelling of the second portion is smaller than a degree of swelling of the first portion, where the degree of swelling is defined by equation (1):
Degree of swelling=(thickness after swelling)÷(thickness before swelling). (1)

2. The elongated medical device according to claim 1, wherein:
the swellable coating layer further comprises a third portion that is disposed closer to the proximal end side than the second portion is to the proximal end side;
the thickness of the second portion after the swelling is greater than a thickness of the third portion after the swelling; and
a degree of swelling, defined by the equation (1), of the third portion is smaller than the degree of swelling of the second portion.

3. A method for producing an elongated medical device including an elongated body which extends from a distal end side to a proximal end side, the method comprising:
a step of preparing the elongated body in which a first elongated body component (I) and a second elongated body component (II), which is disposed on a proximal end side of the first elongated body component (I), are directly or indirectly contiguous with each other, wherein
the first elongated body component (I) is made of a first resin (I); and
the second elongated body component (II) is made of a second resin (II) having a smaller degree of swelling with respect to a first solvent (A) than the first resin (I); and
an application step of applying a first coating material containing a cross-linking agent and the first solvent (A) onto at least a part of surfaces of the first elongated body component (I) and the second elongated body component (II).

4. The method for producing the elongated medical device according to claim 3, wherein, in the application step, a swellable coating layer is formed by applying the first coating material, wherein the first coating material further comprises a hydrophilic polymer

5. The method for producing the elongated medical device according to claim 3, wherein:
in the application step, a base layer is formed by applying the first coating material, wherein the first coating material further comprises a base polymer; and
the method further includes, after the application step, a step of forming a swellable coating layer by applying a second coating material containing a hydrophilic polymer and a second solvent (B) onto at least a part of a surface of the base layer on a side opposite to the elongated body.

6. The method for producing the elongated medical device according to claim 3, wherein:
in the application step, an adhesion layer is formed by applying the first coating material; and
the method further includes, after the application step, a step of forming a swellable coating layer by applying a second coating material containing a hydrophilic polymer and a second solvent (B) onto at least a part of a surface of the adhesion layer on a side opposite to the elongated body.
